(19) United States
(12) Patent Application Publication
(10) Pub. No.: US 2013/0210031 A1
(43) Pub. Date: Aug. 15, 2013

(54) DETECTION OF ADENYLATE CYCLASE

(71) Applicants: Anne E. Boyer, Atlanta, GA (US); Renato C. Lins, Scottdale, GA (US); Zsuzsanna Kuklenyik, Lawrenceville, GA (US); Maribel Gallegos-Candela, Atlanta, GA (US); Conrad P. Quinn, Lilburn, GA (US); John R. Barr, Suwanee, GA (US)

(72) Inventors: Anne E. Boyer, Atlanta, GA (US); Renato C. Lins, Scottdale, GA (US); Zsuzsanna Kuklenyik, Lawrenceville, GA (US); Maribel Gallegos-Candela, Atlanta, GA (US); Conrad P. Quinn, Lilburn, GA (US); John R. Barr, Suwanee, GA (US)

(73) Assignee: CENTERS FOR DISEASE CONTROL AND PREVENTION, Atlanta, GA (US)

(21) Appl. No.: 13/878,378

(22) PCT Filed: Nov. 8, 2011
(86) PCT No.: PCT/US11/59739

§ 371 Date: Apr. 8, 2013

Related U.S. Application Data
(60) Provisional application No. 61/411,056, filed on Nov. 8, 2010.

(51) Int. Cl. G01N 33/569

(52) U.S. Cl. CPC G01N 33/56911
USPC 435/7.4; 435/34; 435/287.2

(57) ABSTRACT

One major problem in diagnosis methods presently available for anthrax is that these methods require several days to produce a result, are rendered unusable after antibiotic use, or are not quantifiable. The only existing treatment for anthrax requires administration soon after infection at a time when patients are exhibiting only mild flu-like symptoms. Thus, by the time a diagnosis is made a patient may be days beyond the time when treatment would be effective. The present invention reduces diagnosis time to as little as four hours providing same day identification of anthrax radically increasing the odds of delivering proper treatment and patient recovery. The rapid identification of anthrax edema factor activity exhibited by the invention is also amenable to in vivo screening protocols for the discovery and development of anthrax vaccines, anti-toxins and edema factor inhibitors. The invention isolates and concentrates edema factor and edema toxin from nearly any sample. By capitalizing on the adenylate cyclase activity of edema factor the invention amplifies output signals producing reliable detection of low concentrations of edema factor previously unachievable. The invention involves novel purification and detection techniques and substrates for rapid, reproducible, and quantitative measurements of anthrax edema factor, and other adenylate cyclases in biological samples.

### CROSS-REFERENCE TO RELATED APPLICATIONS

This application is a U.S. National Phase of PCT/US2011/059,739, filed Nov. 8, 2011, which claims priority to U.S. provisional patent application 61/411,056, filed Nov. 8, 2010, both of which are incorporated herein in their entirety.

### GOVERNMENT INTEREST

The invention described herein may be manufactured, used, and licensed by or for the United States Government.

### FIELD OF THE INVENTION

The invention relates generally to disease diagnostics, and in particular to methods for detecting infection of anthrax in a patient and screening anthrax therapeutics.

### BACKGROUND OF THE INVENTION

Anthrax is caused by infection with Bacillus anthracis, a spore-forming, rod-shaped bacterium. The dormant spore-form is highly resistant to extreme conditions, high temperatures, and a variety of chemical treatments. The spores gain entry either through an open wound, causing cutaneous disease, or by ingestion, causing gastrointestinal disease or are inhaled causing inhalation anthrax. All three forms can progress to a systemic infection leading to shock, respiratory failure, and death. (Mock, M. and Mignot, T. (2003), Cell Microbiol., 5(1):15-23). The stability of the spores and their infectious capacity make them a convenient bioterrorist weapon.

The two known toxins of B. anthracis are binary combinations of protective antigen (PA), named for its ability to induce protective immunity against anthrax, with either edema factor (EF) or lethal factor (LF). PA is the cell binding component of both toxins and is responsible for bringing the catalytic EF or LF into the host cells. EF is an adenylate cyclase which converts ATP to cyclic AMP and causes edema (Brossier, F. and Mock, M. (2001), Toxicon. 39(11):1747-55). The combination of PA-EF forms edema toxin (ETx) which causes edema when injected locally. LF is a zinc-dependent endoprotease known to target the aminotetininus of the mitogen-activated protein kinase kinase (MAPKK) family of response regulators (Id.). The cleavage of these proteins disrupts a signaling pathway and leads to cytokine dysregulation and immune dysfunction. LF combined with PA forms lethal toxin (LTx) which is lethal when injected on its own. It is also known that there are fatal anthrax cases where administration of antibiotics and clearance of bacteria have failed to rescue the patient. This indicates that there may be a “point of no return” level of LTx in the blood that may predict the outcome of infection. Clearly, LTx and its components are important targets for diagnostics and quantification.

Assays for EF activity such as competitive enzyme assays (Duriez, E, et al., Anal. Chem., 2009; 81:5935-5941) or radiometric assays (Gottle, M, et al., Biochemistry, 2010; 49:5494-503), are impractical for high-throughput screening of compound collections and rapid diagnosis of host infection. Methods for rapid screening of patients in a hospital setting or identification of potent and selective EF inhibitors requires an assay that is less labor intensive, has faster turnaround, and is effective at low levels of enzyme.

Development of a safe and effective vaccine for inhalation and other forms of anthrax infection is vital to the health and safety of the population and an essential component of any bioterrorism defense strategy. Additionally, the identification of targeted therapies following anthrax infection is essential to managing a patient population. As such, there exists a need for methods to rapidly identify possible candidate vaccines and treatments. There also exists a need for rapid diagnosis of anthrax infection that can be distinguished from other infections that initially display similar symptoms.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

A method is provided for detecting the anthrax edema factor activity in a sample. Edema factor is isolated and optionally concentrated from the sample. The edema factor (EF) is subsequently reacted with an adenylate cyclase substrate to yield a small molecule reaction product detectable by one of several methods known in the art such as mass spectrometry. As such, relative catalytic efficiency of the edema factor is measured.

The EF is detected in a sample, such as a biological sample, that is acquired by standard methods known in the art from a patient or other test subject illustratively including humans and other mammals. A sample optionally is whole blood, plasma, serum, extracellular fluid, cytosolic fluid, pleural fluid, ascites, tissue, or combinations thereof.

A target form of EF, such as EF, or PA-EF, is isolated and concentrated from the biological sample in an exemplary step through binding to a binding agent specific for the EF or PA-EF, such as beads coupled with an antibody specific to EF. The beads are optionally magnetic, thereby allowing for gentle and rapid separation from other components present in the sample. The isolation and purification optionally occurs on a solid substrate or other substrates known in the art. A solid substrate is illustratively a microtiter plate. Magnetic beads are optionally coated with protein G and an antibody or other molecule specific to the EF or PA, illustratively PA63 or a protein designed to mimic a natural ligand. Antibodies operative herein illustratively include those derived from organisms including a mammal such as a human, mouse, rabbit, monkey, donkey, horse, rat, swine, cat, chicken, goat, guinea pig, hamster, or sheep. The antibody selected is appreciated to be monoclonal or polyclonal. Some embodiments include both monoclonal and polyclonal antibodies. Antibodies specific for various targets are employed illustratively including anthrax protective antigen, lethal toxin, edema toxin, lethal factor, edema factor, or combinations thereof.

Following isolation and concentration, EF is reacted with a substrate for adenylate cyclase such as a nucleotide triphosphate substrate including an adenylate cyclase substrate or a derivative thereof to determine the enzymatic activity specific for EF present in the sample. A substrate is illustratively adenosine triphosphate (ATP). The substrate is optionally tagged with one or more reporter molecules to facilitate detection, the reporter molecule illustratively including a fluorophore, a fluorescence quenching molecule, or a light-absorbing molecule, heavy atom, other reporter molecule that may facilitate detection know in the art, or combinations thereof.

Several detection methods are operable to detect the product of an enzymatic reaction with EF illustratively including mass spectrometry, enzyme assay, luminescence, fluorescence, light absorption, high-performance liquid chromatography, immunoassay, colorimetric assay, and combinations thereof.

An apparatus is also provided for isolating and detecting EF that includes isolation and concentration of EF by binding to magnetic beads. The apparatus includes a reaction chamber in which the isolated and concentrated EF is reacted with a substrate to produce a product that is subsequently detected by mass spectrometry or other detection method.

### BRIEF DESCRIPTION OF THE DRAWINGS

FIG. 1 is a schematic of Bacillus anthracis binary toxins where activated protective antigen (PA₆₃), which is responsible for binding cell surface receptors, binds to lethal factor (LF) forming lethal toxin (LTx) and edema factor (EF) forming edema toxin (ETx), and where edema factor is an adenylate cyclase (AC) that converts adenosine triphosphate (ATP) and other nucleotide triphosphates to cyclic adenosine monophosphate (cAMP) or other cyclic nucleotide monophosphates, and where toxins may exist in the monomer form EF and LF or in complex with PA, LTx and ETx;

FIG. 2 is a schematic of various methods for the isolation and optional concentration of ACs from a biological sample;

FIG. 3 is a schematic of a process for isolation and concentration of AC from a biological sample and detection by mass spectrometry according to one embodiment of the invention;

FIG. 4 is a schematic of the enzymatic activity of an adenylate cyclase enzyme using ATP as a substrate;

FIG. 5 is a schematic of several AC reactions from concentrated ACs present on beads;

FIG. 6 is a schematic of an AC enzymatic reaction and mass spectrometric detection of the substrate ATP and product cAMP;

FIG. 7A illustrates a standard curve based on EF concentrations spiked in plasma versus chromatogram peak area ratios for the enzymatic product cAMP derived from a capture and enzymatic reaction assay using isolated EF;

FIG. 7B illustrates several chromatograms for cAMP derived from capture and reaction of decreasing levels of EF with substrate ATP illustrating a LOD of 16 fg/ml EF;

FIG. 8 illustrates embodiments of inventive processes used to detect the presence of EF in the serum of control and B. anthracis infected Rhesus macaques prior to infection (A), at day-2 following infection (B), and at day-4 following infection (C).

FIG. 9 is a schematic of one embodiment of a process of isolating and detecting EF in a sample.

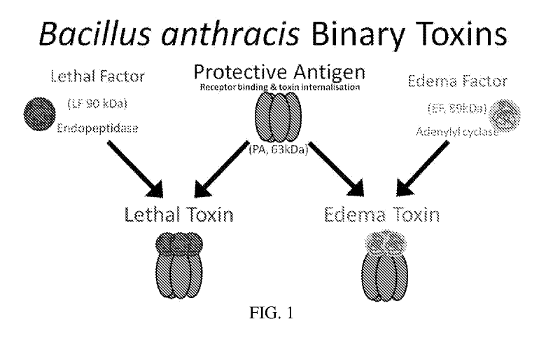

### DETAILED DESCRIPTION OF THE EMBODIMENTS OF THE INVENTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the process is described as an order of individual steps or using specific materials, it is appreciated that described steps or materials may be interchangeable such that the description of the invention includes multiple parts or steps arranged in many ways as is readily appreciated by one of skill in the art. Although the specification is specifically directed to detection of B. anthracis, the processes described herein are similarly operable for detection and quantification of any adenylate cyclase from any other source. Another specific source of an adenylate cyclase includes Bordetella pertussis. As such, the processes described herein are equally appreciated as applicable to detection of infection by Bordetella pertussis, of for the detection, discovery, or identification of therapeutics or vaccines for the treatment and/or prevention of Bordetella pertussis, Bacillus anthracis, other biological infective agent that produces a specifically isolatable adenylate cyclase, or combinations thereof. It is further appreciated that the exemplary adenylate cyclase is EF, that EF is substitutable by any other adenylate cyclase from any other source. As such, the term EF as used herein is intended to mean edema factor as well as any other adenylate cyclase.

Processes and substrates are provided to rapidly and reliably recognize infection by B. anthracis in a human subject or other animal, or in the environment. Methods are provided for rapidly isolating and optionally concentrating anthrax edema factor (EF) and then efficiently detecting the activity of EF as a marker of B. anthracis infection in a subject.

As used herein, the term “isolated” or “purified” is defined as substantially free of cellular material or other contaminating proteins from a sample from which the target adenylate cyclase is derived, or is substantially free of chemical precursors or other chemicals when chemically synthesized. The language “substantially free of cellular material” includes preparations of a protein in which the protein is separated from cellular components of the cells from which it is isolated or produced. Thus, a protein that is substantially free of cellular material includes preparations of the protein having less than about 30%, 20%, 10%, 5%, 2.5%, or 1%, (by dry weight) of contaminating material. In some embodiments of the present invention, an EF is isolated or purified. The term isolated or purified optionally is exclusive of other components of a complex in which the EF is bound, illustratively, PA-EF complexes. In such embodiments, isolated EF is inclusive of EF-PA or other EF complex components.

By capitalizing on the enzymatic activity of the EF to cleave adenylate substrates suitable for rapid detection and quantitation in a hospital or laboratory setting, the present invention has utility as a diagnostic test that guides patient treatment of B. anthracis infection. The inventive test is rapid, highly sensitive and specific. The invention also has utility in monitoring ETx toxemia. The invention affords a process to monitor onset, progression, and response to treatment kinetics of B. anthracis infection, including the effectiveness of anthrax therapeutics. As such, the invention is also useful as a screening assay for identification of therapeutics suitable for the treatment of B. anthracis infection in a subject.

Monitoring disease progression in a patient population is essential to providing optimal treatment following infective exposure to B. anthracis. Often distinguishing an infection by B. anthracis from other flu-like or febrile illnesses is difficult, particularly in a setting where exposure to B. anthracis is rare or the threat of bioterrorism is low. Early detection and identification of B. anthracis exposure in a bioterrorism situation is a benefit in tracking the infection source. Currently employed diagnostic techniques for identifying B. anthracis infection are ineffective within 24 hours after antibiotic use, require lengthy incubations, or do not quantify infection or toxemia. The present inventive processes employ several levels of specificity illustratively specific immunoisolation of EF or ETx and substrates that are highly specific for EF. While similar detection of lethal factor (LF) by enzymatic methods have observed detection within 12 to 24 hours post exposure to B. anthracis spores, the detection limit of the current inventive methods for EF are 50-100 times lower than that for LF. Thus, the invention has the capability of detecting B. anthracis infection within hours after spore exposure. It is appreciated that the present invention offers results within 4 hours of obtaining a biological sample such that directed treatment strategies may begin earlier, enhancing potential patient survival.

In some embodiments, a sample is obtained from a patient or test subject and immediately analyzed or alternatively frozen or otherwise stored for later analysis at the sites of collection or remote from the source of the sample. A non-limiting example includes samples taken in environments lacking state of the art diagnostic instruments. As an illustration of some embodiments, a simple blood sample is drawn into vacutainer or other tubes known in the art such as by venipuncture and then immediately frozen for prompt shipment. As a result, a diagnosis of infection is obtained in as little as 4-24 hours following a patient presenting symptoms of exposure to B. anthracis.

It is appreciated in that the present invention is also applied to screening foodstuffs for human or other animal consumption. Wild animals or farm animals become infected by B. anthracis from grazing in areas contaminated by anthrax spores or from food that is unintentionally or intentionally contaminated with the bacteria. Natural anthrax exposures in people occur from contact with contaminated animals or animal products. Human anthrax exposures more rarely occur from intentional contamination of products or areas that humans might contact (e.g. mail). Biological samples from meat processing plants are rapidly screened for prior exposure to anthrax by the processes of the invention.

It is appreciated that a sample is optionally a biological sample, an environmental sample, or other sample. A biological sample illustratively includes whole blood, plasma, serum, extracellular fluid, cytosolic fluid, or tissue and other fluids known to harbor the bacterial toxins, the bacteria, or samples from individuals thought to have previously contained the bacteria. Simple techniques known in the art may be employed to homogenize, liquefy, or otherwise process the sample for analysis by the present invention. In instances when subject or source is sampled, the sample is amenable to being frozen and analyzed remotely in time and place. Alternatively, an inventive field kit is employed.

The processes described herein are also amenable to determining the presence or absence of bacteria such as B. anthracis in an environmental sample. An environmental sample is illustratively soil, water, physical source such as mail, or other non-biological source. Environmental samples are illustratively analyzed by the present inventive process for the presence of B. anthracis. Direct soil samples are used or “incubator” cells may be employed to provide a system by which exposure may be studied.

Processes are performed using one or more of numerous biological samples illustratively including whole blood, plasma, serum, extracellular fluid, cytosolic fluid, or tissue. In some embodiments, serum or whole blood is used as a biological sample due to the ease in obtaining a sample by a venous blood draw from a patient or other test subject. It is recognized in the art that numerous other sample types are suitable in the present invention dependent on the application desired. By way of example, a sample may be as simple as an aqueous buffering agent such as HBS or PBS, any of which are spiked with known or unknown levels of EF. Cell growth media is also suitable as a sample for screening transfected cell cultures for expression of active EF according to the present invention. It is appreciated that other biological samples are used such as a homogenized tissue sample that may or may not have been infected with anthrax.

Numerous species are suitable as sources of samples for use in the inventive processes. As used herein, a “host”, synonymously described herein as a patient or subject, is any organism able to sustain Bacillus anthracis bacteria or may harbor EF and specifically includes non-human primates, such as monkeys, baboons, chimpanzees and gorillas; humans; ruminates such as sheep, cows and goats; murine such as rats, mice, and other murine; equine such as horse, donkey, and other equine; bovine; and rabbits.

Inventive processes are also operative as a diagnostic tool to identify and monitor the progression of infection by anthrax spores such as may occur following a bioterrorist attack. However, it is recognized in the art that the invention is used in numerous other types of analyses illustratively including screening for suitable vaccines and for efficacy of therapeutics in an in vitro or in vivo screening assay where the source of the EF may be transfected protein expressing cell lines. Another non-limiting use is the screening of cattle that that have been found dead on a ranch such that the remainder of the herd may be rapidly and properly isolated from any infected animals reducing the impact of a disease outbreak.

Upon selection of a sample, detecting EF involves isolating and optionally concentrating EF from a biological sample. It is appreciated that EF is optionally free EF, or EF that is incorporated into a larger complex such as EF bound to PA. Optionally, beads, illustratively, nonporous magnetic beads coated with protein-G and bound to antibodies that recognize and bind EF or PA-EF, are employed to capture the EF from the sample. Magnetic beads have the advantage of requiring no centrifugation, thus, allowing magnetic bead regeneration without loss of binding capacity. Magnetic beads also allow for minimal loss of sample due to pipetting as magnetic beads migrate to the sides of the reaction tube. It is further appreciated that magnetic beads allow for small scale isolation methods minimizing biological sample requirements. Other bead types or compositions operative herein illustratively include agarose, sepharose, nickel, or other materials known in the art. Numerous commercial sources are available for protein purification beads including Invitrogen, New England Biolabs, Quiagen and Bachem.

Protein-G or tosyl activated coated magnetic beads are prepared and reacted with a suitable antibody for recognizing and binding EF or another member of a complex of which EF is a member. Monoclonal antibodies, polyclonal antibodies, or combinations thereof are suitable antibodies. Optionally, monoclonal antibodies are used that recognize a region on EF that does not result in interference with the adenylate cyclase (AC) activity of the toxin. The antibodies are readily derived from numerous organisms including, but not limited to a human, mouse, rabbit, monkey, donkey, horse, rat, swine, cat, chicken, goat, guinea pig, hamster, or sheep. Antibodies specific for EF are readily obtained from numerous commercial sources including Santa Cruz Biotechnology, Santa Cruz, Calif. Anti-EF antibodies may be reacted with protein-G coated beads such that the antibodies are bound to the beads. It is appreciated that antibodies directed to ETx such as those described by Albrecht, M T, et al., Infection and Immunity, 2007; 75:5425-5433 directed to PA or antibodies directed to Bordetella pertussis adenylate cyclase (PAC) such as antibodies described by Lee, S J, et al, Infection and Immunity, 1999; 67:2090-2095 are similarly employed in conjunction with, or as an alternative to anti-EF. It is appreciated that the inventions are operable toward any adenylate cyclase (AC) or the detection of any AC expressing organism, or the detection of any AC in any biological sample. These antigen targets of antibodies are collectively defined as AC or EF. The beads are then blocked with bovine serum albumin (BSA), polyethylene glycol (PEG), or other blocking agents known in the art. A sample is incubated with the antibody coated beads for sufficient time to allow equilibrium binding to develop, generally between 1 minute and 3 hours depending on the affinity of the antibody, the incubation temperature, and the anticipated concentration of EF in the biological sample. EF bound beads are then washed with a suitable buffer such as PBS-T, HBS-PEG, or other suitable buffering system known in the art to remove any unbound protein or other serum or sample components. An appropriate EF substrate is added to the washed beads and incubated between 1 minute and 20 hours, and typically about 2 hours. However, it is recognized in the art that the appropriate incubation time depends on substrate affinity, kinetic or catalytic efficiency constants intrinsic to the selected substrate such that a detectable amount of product is formed in the incubation time. Such constants are readily determined by techniques well known and commonly practiced in the art.

Substrates operative in the present inventive process are selected based on known affinity and kinetic constants as well as by the method of detection to be employed under the inventive processes. The selected substrate mimics the natural target of an adenylate cyclase or is a natural target of EF depending on the assay detection method to be employed. Optionally, the selected substrate is a nucleotide triphosphate, illustratively, ATP or a derivative of ATP that is operable as an EF substrate. Several derivatives are operable herein illustratively including: [aV]Ah1P-PNP described by Krishna, G. et al, J. Biol. Chem., 1972; 247:2253-2254; [p1NH]ppA, described by Limberd, L E, Biochem J., 1981; 195(1): 1-13; and adenylyl imidodiphosphate (AMP-PNP) described by Bagger-Sjöbäck, D, et al., European Archives of Oto-Rhino-Laryngology, 1980; 228: 217-222; along with other substrates known in the art. It is well within the skill of the art to determine whether a derivative of ATP is a substrate for EF by simple incubation of the substrate with a purified AC such as EF or PAC and determining whether product is formed.

The cAMP or other cyclized reaction product resulting from reaction of EF with the substrate is optionally derivatized prior to detection. An optional agent for derivatization is 2-chloroacetaldehyde essentially as described by Zhang, L, et al., Int. J. Mol. Sci. 2006; 7:266-273. Other agents for derivitization that provide improved detection such as by altering the ability of the reaction product to be successfully ionized in a mass spectrometer or to be detected such as by fluorescence are known in the art and are operable herein.

The inventive processes are amenable to numerous detection protocols and apparatuses. Illustrative examples include mass spectrometers, fluorometers, chromatography systems, coupled enzyme assays, competitive enzyme assays, among others known in the art. Any method suitable for detection of a cyclized nucleotide or derivative reaction product or a derivative thereof is operable herein. In some embodiments, a sample of the analyte is analyzed by mass spectrometry such as ESI-MS alone or coupled with liquid chromatography (LC). ESI-MS has the advantage of being readily coupled to LC for rapid and robust separation of product from substrate and its subsequent detection and quantification. It is recognized in the art that numerous other forms of mass spectrometry may be employed as detection methods in the present invention such as MS/MS, etc.

Analysis is optionally performed by liquid chromatography (LC) coupled to mass spectrometry (MS). LC techniques suitable for use in the invention illustratively include high-performance LC, or ultra-high performance LC techniques. Examples of suitable columns for separation of products and residual substrate illustratively include a weak anion exchange column available from Thermo Scientific such as the Biobasic AX column from Thermo Scientific Inc. (Waltham, Mass.). Examples of suitable mobile phases illustratively include a 90-20% acetonitrile and pH 6.5-10 eluent gradient. It is appreciated that other column types and mobile phase systems are similarly suitable for use in the present invention. Column parameters such as inner diameter, length, number of theoretical plates, etc. are recognized in the art and persons having ordinary skill in the art readily recognize methods of optimizing these and other necessary parameters to facilitate effective separation reaction products. Thus, it does not require undue experimentation to adjust parameters of LC columns.

A second or other additional column is optionally employed to further separate the products and any residual substrate or other contaminant. In some embodiments, the elution of an HPLC column is coupled to a second chromatographic step. The separated products and residual substrate (if any) are illustratively subsequently submitted to a mass spectrometry system for detection, identification, and quantification.

Suitable detection and quantitation systems illustratively include electrospray, time of flight (TOF), multiple quadrupole, and other types of mass spectrometry systems known in the art. Illustratively, a Waters Q-Tof Premier TOF quadrupole tandem mass spectrometer available from Waters, Corp. or an API 4000-Q trap triple quadrupole tandem mass spectrometer (Applied Biosystems, Foster City, Calif.) are each suitable for use in the instant invention. It is appreciated that other brands and types of mass spectrometers are similarly suitable.

Simultaneous with or subsequent to identification and detection in a suitable detector, the amount of product is quantified such as by reference to a standard. A standard is optionally an internal standard or a standard curve previously or simultaneously generated.

Suitable mass labels are optionally incorporated into substrates or to products through derivatization illustratively include incorporation of deuterium, ³H, ¹³C, ¹⁵N, fluorine, florescent labels such as rhodamine, Oregon green or others known in the art, radioactive labels, mass labels as described in U.S. Pat. No. 6,649,354, those described in WO/1998/026095, and others known in the art.

Screening inhibitors of ACs such as EF and PAC in vivo provides physiologically relevant information as to the potency, bioavailability, rate of clearance, and efficacy of potential small molecule or antibody based inhibitors of one or more adenylate cyclases. The present invention is particularly useful as a rapid, high-throughput assay format for screening such inhibitors. In some embodiments the detection limit using LC/MS analyses is 16 fg/ml EF or less.

Antibodies are available or are raised against EF from numerous species commonly used for screening purposes such as murine, rabbit, guinea pig, hamster, canine, swine, or monkey. Techniques for raising antibodies to molecular targets are well known in the art.

Non-limiting examples of screening protocols using processes such as those described herein include early in vivo screening protocols employing mice dosed with small molecule or other compounds. The mouse is optionally subjected to inhalation anthrax or other forms to initiate an onset of infection. Following administration of a lead compound directed toward preventing anthrax infection, proliferation, ability of toxins to bind to target cells, or other mechanisms of anthrax infection, small blood samples are acquired and analyzed by the present invention for EF activity. As the present invention requires only modest sample quantities such as 5 μl volumes, numerous time points are readily obtained from a single mouse allowing for in vivo kinetic measurements.

The present invention is also employed in screening protocols for the identification and trials of candidate vaccines by allowing rapid observation of the degree to which antibodies generated by a vaccine neutralize catalytic activity associated with a given AC.

As the present invention capitalizes on the activity of AC in a biological sample, it is operative to predict disease progression in animals that have been subjected to Bacillus anthracis infection or Bordetella pertussis infection that may or may not have been pretreated with a vaccine candidate. A correlation is expected between the efficacy of a vaccine and reduced levels of isolated target AC present in a biological sample from a test host. As such, sampling host tissues or fluid samples following the initiation of infection provides a real-time readout of the progress of the infection. A reduction of the levels of infection specific AC activity in a host treated with a vaccine serves as a direct measure of vaccine efficacy. Thus, the present invention has numerous advantages over simple death screening models as it is expected to provide a superior correlation with lower levels of vaccine efficacy providing investigators with data that allows for more complete differentiation between possible candidate vaccines. It is further appreciated that numerous additional parameters of protection may be analyzed using the present invention. The present invention is well suited to determine alterations in the rate of infection progression, levels of free EF, levels of ETx, other ACs, rate of disease resolution, or other parameters common to the art to screen, differentiate, or monitor vaccine performance.

While the present invention detects EF at 16 femtograms (fg) per milliliter or less, it is appreciated that the subject invention is readily practiced with a larger sample toxin quantity, volume, or both. It is appreciated that low concentrations of EF are detectable in a sample. Illustratively, an EF is detectable to levels of less than 1 pg/ml. Optionally, levels of detection are less than 1 pg/ml to less than 5 fg/ml, or any value therebetween. The ability of specific antibodies coated on beads may be used to isolate EF, ETx, PAC, or other ACs in large sample volumes such as from wet soil by simple agitation of the sample such that the isolation agent (e.g. beads coated with antibody) remain in suspension for the isolation period. In this way even dilute biological samples may be screened for the presence of anthrax or other AC expressing organisms.

Kits for the detection of EF, diagnosis of infection, or screening are also provided. An inventive kit optionally employs prepackaged anti-EF or anti-ETx coated beads, or other isolation agent, to isolate EF from a biological sample. A reaction chamber is optionally provided for isolation. Buffers are optionally included with the kit to be illustratively used for washing the beads, diluting the sample, eluting the beads, reacting with the substrate, reconstituting the substrate, storing the beads, storing the substrate, freezing or otherwise storing the isolated and concentrated EF, freezing or otherwise storing the EF reaction products, preparing samples for detection, or combinations thereof. Suitable buffers illustratively include phosphate buffered saline (PBS), phosphate buffered saline plus Tween-20 (PBS-T), HEPES buffered saline (HBS), HBS-Tween-20 (HBS-T), citrate-phosphate buffers, water, or other suitable buffer(s) known in the art. The reaction chamber is optionally used for conversion of substrate to a cyclized reaction product. Optionally, a second or additional reaction chamber is provided for reaction with additional substrate. The isolated EF is amenable to freezing and shipment for remote analyses. It is further appreciated that products are also amenable to freezing for later detection, quantification or analysis at a remote location and time. These or other methods of employing the present invention may be used to deliver rapid, effective diagnosis on a worldwide scale in a time frame that is not possible with current diagnostic techniques.

The present invention is further detailed with respect to EF and PAC purification and detection as representative of the present invention detection of other ACs. The following examples are not intended to limit the scope of the claimed invention and instead provide specific working embodiments.

### EXAMPLES

### Example 1

Preparation of Tosyl-Activated Magnetic Beads.

Tosyl-activated magnetic beads are obtained from Invitrogen. 20-100 μl of bead suspension are used to covalently link immunoglobulin (IgG) from a 100 μl sample containing IgG to the beads according to the manufacturer's protocol. To separate the beads, the reaction tube is placed on a magnet for 1 min and the resulting supernatant discarded by aspiration. The beads are resuspended in phosphate buffered saline with 0.05% Tween20, pH 7.3 (PBS-TW) and stored until ready for use. Thorough washing is achieved by repeating the magnetic pelleting and resuspension steps three times

### Example 2

Coating Tosyl-Activated Beads with Desired Anti-EF or Anti PAC Antibody.

Anti-EF, anti-PA, or anti-PAC (or other antibodies) are coated onto magnetic beads forming magnetic antibody beads (e.g. MABs). EF or PAC-specific MABs are prepared using mouse monoclonal anti-EF IgG or anti-PAC IgG according to the manufacturer's protocol (Invitrogen) using 40 μg IgG/100 μl magnetic bead suspension.

### Example 3

Purification and Concentration of EF from Serum.

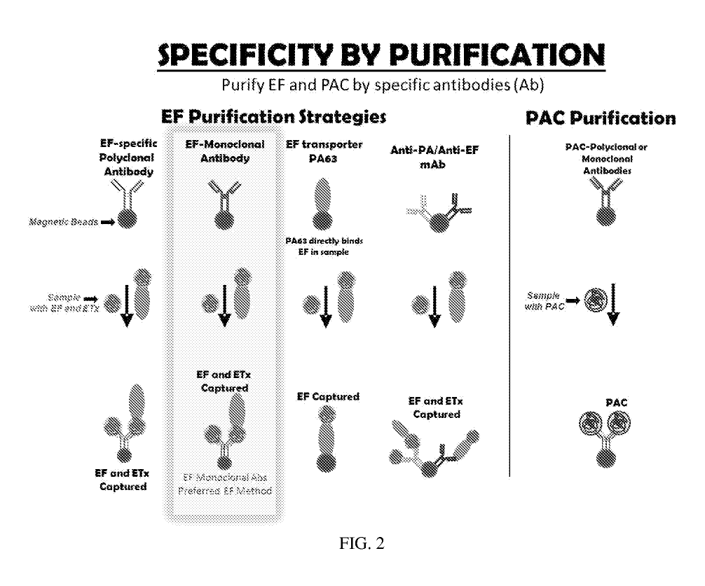

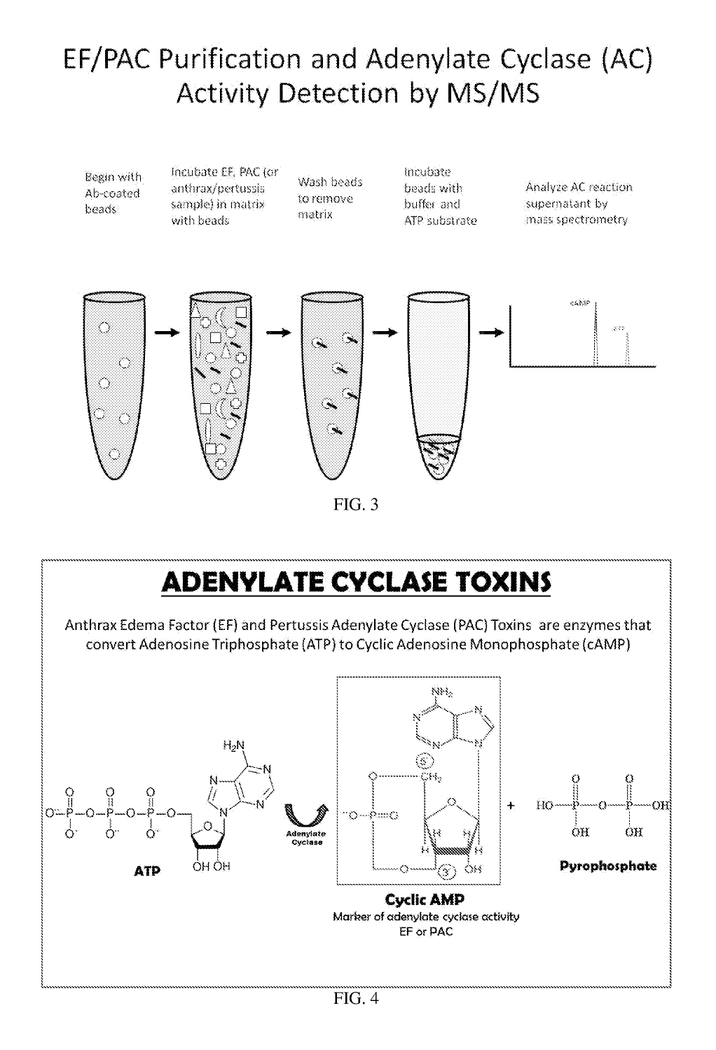

A serum, plasma, pleural fluid or other biological sample is obtained from a patient or infected animal. The sample is diluted 1:5 in 500 or 1000 μl PBS-TW and mixed gently with 20 μl EF MABs for 1 hour. The beads with EF and/or ETx bound antibody are retrieved, washed three times in PBS-TW and reconstituted in PBS-TW for further analyses by enzymatic reaction and mass spectrometry, as shown in FIGS. 2 and 3.

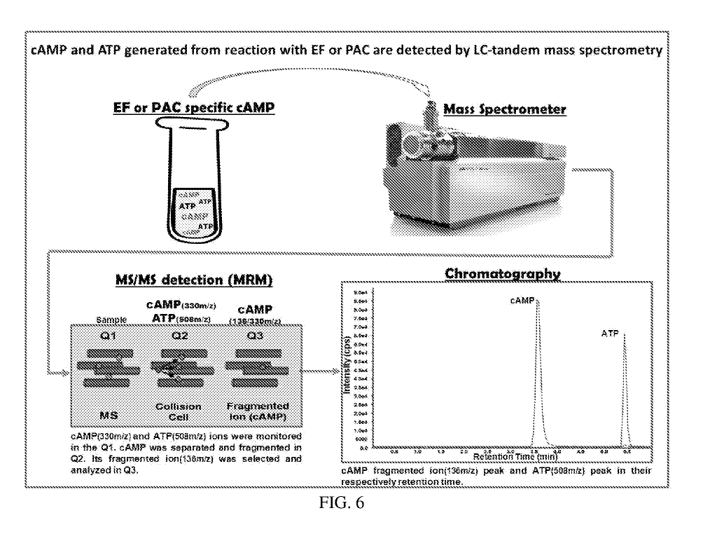

One approach for isolation of EF uses protective antigen (PA) antibody (anti-PA IgG) on MABs for total EF (EF+ETx) retrieval. The first step begins with addition of free activated PA63 that binds to free EF converting it into complexed form, ETx, rendering all EF as the complexed form ETx. Then a PA-MAB that is specific for the distal cell receptor binding portion of PA63 as depicted in FIG. 2 where the antibody binds to PA63 remote from the PA-EF (ETx) interface is used to capture the total EF as converted to ETx. The PA-MAB bound ETx is then reacted with an ATP in the presence of calmodulin, and the enzymatic activity of EF in complex as ETx is detected by mass spectrometry, as shown in FIG. 6.

Alternatively, or in addition, one or more EF mAbs is used to capture total EF (free EF+ETx). This requires that the EF mAb bind an EF antigen epitope distinct from the interaction interface between the PA and EF. The EF mAbs capture both free EF and EF in complex with PA as ETx for total EF (free EF+ETx) which is then exposed to an EF substrate ATP and calmodulin producing EF specific enzymatic reaction products which are detected by mass spectrometry. For example, 5 pg of EF are retrieved with EF MABs without affecting the ability of EF to enzymatically cyclize the ATP substrate. EF (5 pg) complexed with PA (PA-EF or ETx) is also easily retrieved with EF MABs and reacted with buffer, calmodulin, and substrate ATP, for ≦2 hours at ≧30° C. consistent with the procedures detailed in the above examples. ETx containing 5 pg of EF is retrieved with PA MABs consistent with the above examples followed by mixing with buffer, calmodulin, and ATP, and incubated for 2 hours at ≧30° C. The protocols are able to purify EF and ETx with either EF MABs or PA MABs.

### Example 4

On-Bead Substrate AC Reaction.

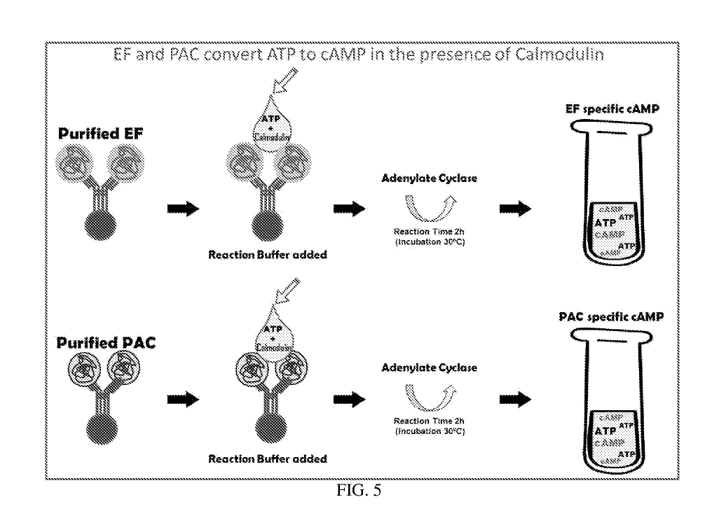

Purified EF or PAC are incubated at 30° C. for 2 h in an optimized AC reaction buffer (MgCl₂ (40 mM), EDTA (1 mM), ATP (1 mM), Calmodulin (10 μM), CaCl₂ (10 μM), HEPES (20 mM) and BSA (0.1 mg/mL)). EF or PAC are activated by the cofactor calmodulin, which then allows the enzymes to catalyze the conversion of adenosine triphosphate (ATP) to cyclic adenosine monophosphate (cAMP). The amount of cAMP generated after 2 h incubation is proportional to the amount of EF or PAC captured from a sample. (FIG. 5.)

### Example 5

Off-Bead Substrate AC Reaction.

EF or EF-PA are alternatively ‘not isolated’ from a patient serum sample. Instead, serum is diluted 1:10 directly in reaction buffer, incubated with 1 mM of ATP and 10 μM calmodulin in 200 μl buffer over 2 hours at ≧30° C., and sampled at times 5, 10, 15, 30, 45, 60, 90, 120, and 240 minutes. A small fraction of the reaction mixture is removed for subsequent identification and quantitation by LC/MS/MS as depicted in FIG. 6. Kinetic profiles of the reactivity of EF over time can be determined from patient samples containing EF or from samples containing recombinant EF. This provides a useful protocol (when specificity is already determined) for analyzing known samples with EF and ETx and samples containing recombinant EF or ETx.

### Example 6

Identification of Reaction Products by LC-ESI Mass Spectrometry.

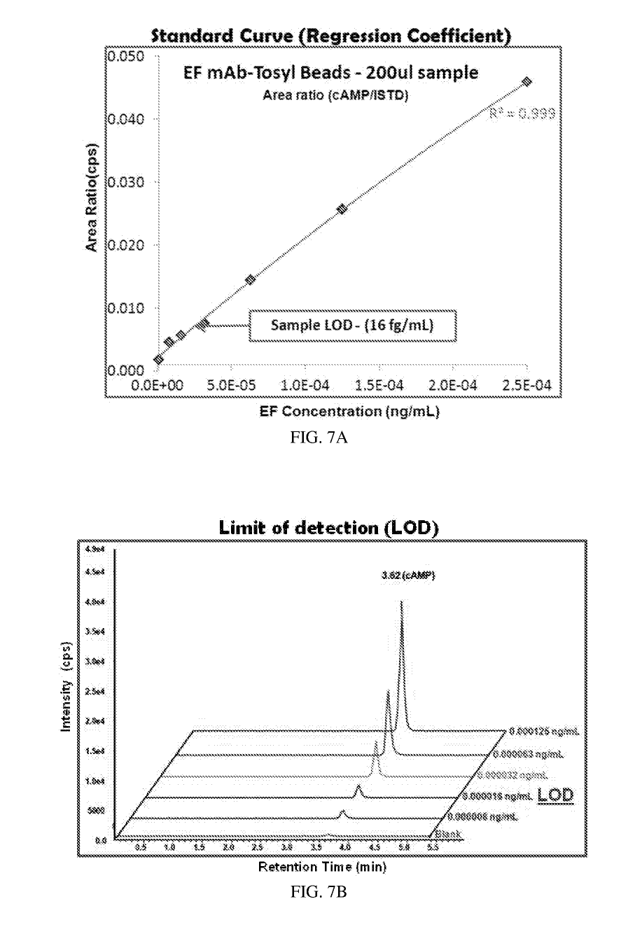

EF, PAC, and/or ETx are isolated from serum of an infected subject as per the above examples and subjected to a reaction using the substrate ATP. The level of cAMP generated is measured by LC/MS/MS as illustrated in FIG. 6. The amount of cAMP generated from incubation with purified EF or PAC is proportional to the amount of EF in a sample. FIG. 7A above shows the ratio of the areas of the cAMP peak/internal standard peak plotted versus EF concentration. The limit of detection (LOD) for 200 μl sample is 0.000016 ng/ml=16 fg/ml or 0.18 attamoles/ml (36 zeptamoles in 200 μl). FIG. 7B illustrates a chromatogram with the cAMP peaks for samples without EF (blank) or with EF above, at, and below the LOD for which the peak area is at least 3 times greater than the blank.

### Example 7

Reactions Performed with PA Specific Antibodies

The antibodies of Table 1 that are either monoclonal antibodies specific for PA or Polyclonal antibodies specific for PA are complexed with Tosyl-activated magnetic beads essentially as described in Example 2. Each of the antibody coated beads, or combinations of antibody coated beads, are used in isolation reactions to isolate and concentrate EF or ETx from serum, plasma, or pleural fluid as described in Example 3.

TABLE 1
Edema Factor Monoclonal Antibodies

| | |
|---|---|
| EF-01 | Mouse IgG |
| EF-02 | Mouse IgG |
| EF-03 | Mouse IgG |
| EF-04 | Mouse IgG |
| EF-05 | Mouse IgG |
| EF-06 | Mouse IgG |
| EF-07 | Mouse IgG |
| EF-08 | Mouse IgG |
| EF-09 | Mouse IgG |
| EF-10 | Mouse IgG |
| EF-11 | Mouse IgG |
| EF-12 | Mouse IgG |
| EF-13 | Mouse IgG |
| EF-14 | Mouse IgG |
| EF-15 | Mouse IgG |
| EF-16 | Mouse IgG |
| EF-17 | Mouse IgG |
| EF-18 | Mouse IgG |

Edema Factor Polyclonal Antibodies

| | |
|---|---|
| Alpha Diagnostic | Goat IgG |
| List Biological | Goat IgG |
| Biodesign | Rabbit IgG |

Each of the antibodies of Table 1 is capable of isolating EF from each tested biological fluid.

The isolated EF complexed with the beads of Table 1 are used to determine the AC activity of the isolated EF as described in Example 5 and detected as in Example 6. Each of the antibodies of Table 1 are capable of isolating EF in sufficient quantities to be detected by the presence of reaction products by LC-ESI-MS.

### Example 8

Determination of EF Activity in Uncomplexed, Complexed PA-EF (ETx), and Antibody Bound Forms of Each.

To determine activity of EF in different states and bound by different antibodies, 5 pg of free uncomplexed EF and 5 pg EF complexed with PA₆₃ (PA-EF or ETx) are each added directly to reaction buffer or are first captured by different MABs then incubated in reaction buffer containing calmodulin and ATP, and incubated for 2 hours at ≧30° C. and the resulting cAMP quantified by LC/MS/MS. Comparing the AC activity of free EF (uncomplexed and unbound) to EF in complex with PA (ETx) and to either free EF or ETx bound by various antibodies provides a measure of how the binding partners (PA or mAb) change the catalytic activity. This method can facilitate screening of mAbs and other anthrax EF or PA directed toxin therapeutics for neutralizing activity. Under like conditions and in the presence of the same substrate, unbound ETx (PA-EF complex) has similar activity to free EF. Different EF MABs produce different EF activities, some similar and some lower than unbound EF alone indicating that some mAbs partially neutralize EF activity and may be good anti-toxin candidates.

### Example 9

Rhesus Macaque Experimental Infection through Inhalation of B. anthracis Spores.

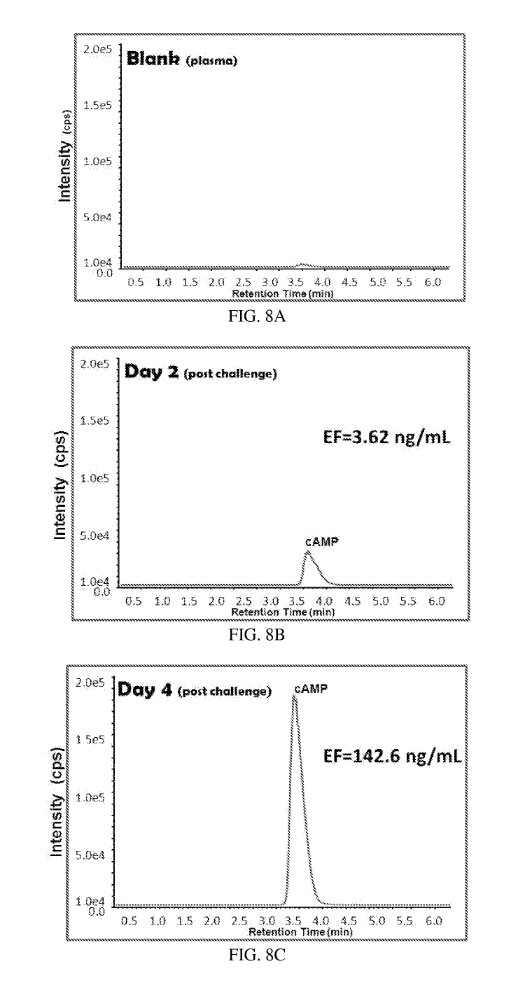

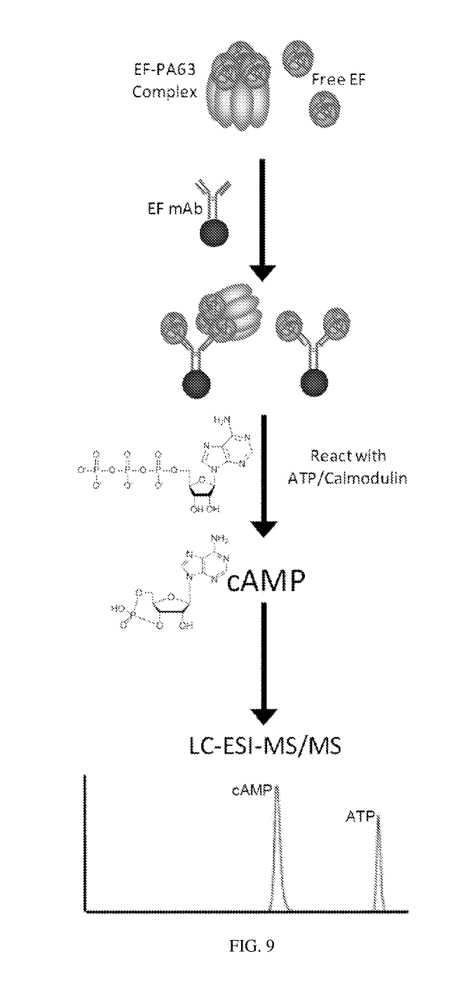

Three rhesus macaques are exposed to experimental infection using airborne spores of B. anthracis and monitored for onset of inhalation infection, rate of infection progression, and correlation with physiological complications. Biological samples of blood serum (1 ml) are taken from each animal prior to exposure to anthrax spores, and on day 2 and day 4. A serum blank and serum from day-2 and day-4 post-challenge are analyzed. The day 4 sample, known to have high LF, is pre-diluted 1:100 then 20 μl of diluted sample is purified along with the 20 pl EF standards. After isolation, EF is incubated in reaction buffer including calmodulin and the substrate ATP, and then the specific cAMP generated is analyzed and quantified by LC-MS/MS, using a 20 μl standard curve ranging from 0.0006 to 10 ng/ml. The resulting levels of cAMP in a serum blank (equivalent to pre-dose), day-2 and day-4 are presented in FIGS. 8A-C respectively.

Results for total LF and LTx (PA-LF complex) have shown that the ratio of LTx:Total LF may define the ‘stage’ of infection (early, middle, and late) and indicate the disease severity and need for advanced therapeutics. This may be true for ETx:EF as well. The inventive method is used to determine specific levels of ETx in rhesus macaque following a similar inhalation mediated infection by B. anthracis. Beads coated with MABs specific for PA that neither interfere with the interaction of PA with EF nor interfere with the catalytic activity of EF with respect to substrates are employed. Comparisons between the levels of total EF (EF+ETx) and ETx are simultaneously obtained with a fraction of the same biological sample at days 2 and 4 giving a ratio of ETx:total EF.

### Example 10

Analysis of Vaccine Efficacy in an Animal Model of B. anthracis Inhalation Infection.

A murine model of vaccine induced protection is employed essentially as described by Peachman K. K. et al. (2006), Infection and Immunity, 74:794-797. Female CBA/J mice (6 weeks old; 15/group) are purchased from the Jackson Laboratory (Bar Harbor, Me.) and maintained with food and water ad libitum. Positive-control mice are immunized by i.m. injection with 20 μg of rPA mixed with alum. Animals are immunized at week 0 and boosted at weeks 2 and 4. Animals are bled at 2-week intervals, and sera analyzed for rPA specific immunoglobulin G (IgG) by ELISA or for toxin-neutralizing antibodies as measured by the dilution of antiserum required for 50% reduction in cellular cytotoxicity (ED₅₀). At week 9 post-immunization the mice are challenged by the intranasal route with 234,000 spores (10-50% lethal doses) of B. anthracis Ames spores administered in a 50 μl volume in the nasal cavity with a pipette. Biological samples of blood serum are obtained at day zero and each day until mortality or a maximum of 35 days post infection. Samples are processed by the present inventive method and subjected to identification and quantification by LC-MS/MS. Prior immunization results in decreased levels of free EF in the serum of both survivors and non-survivors at mortality. However, the levels of free EF will generally be much lower in vaccinated survivors than the deceased group.

### Example 11

Detection and Quantification of EF and ETx in a Human Patient With Inhalation Anthrax.

Biological samples of whole blood, serum, plasma, or pleural fluid is obtained upon hospitalization (day 4 post-symptom onset) or as early as possible following a known or possible exposure to anthrax. Quantification of biological sample EF or ETx levels is performed using the inventive method employing MABs specific for EF or PA and analyzed following isolation and use in an enzymatic reaction including calmodulin and ATP as a substrate. Levels of EF in excess of 10 ng/ml are detected in plasma or serum at day 4 post symptomatic. These levels are confirmed in patient pleural fluid. Plasma/serum samples are obtained each day following hospitalization and levels of EF will decrease with time. Similarly, levels of ETx will be detected at on the first day of hospitalization and will be monitored with time. B. anthracis infection is confirmed using traditional diagnostic techniques 4 days after hospitalization (e.g. day 8, Walsh et al, 2007) indicating that the present inventive method identifies infection at a much earlier time point such that proper treatment may begin sooner increasing chances for survival.

### Example 12

EF Inhibitor Screening in Rabbit Inhalation Infection Model.

Screening of EF inhibitor candidates is performed in rabbits following infection with Ames spores as described by Shoop et al. (2005), PNAS USA, 102:7958-7963. Dutch belted (DB) rabbits (weight, 2 kg; age, 16 wk) purchased from Covance (Princeton, N.J.) are used. Six DB rabbits are dosed s.c. with the EF inhibitor adefovir dipivoxil known to be a potent EF inhibitor as described by Shen, Y., et al., PNAS-USA, 2004; 101: 3242-3247, PGE₂-imidazole, or other inhibitors as described by Chen, D., et al., Bioorg Med Chen?. 2008; 16(15):7225-7233, for 7 days and six rabbits are dosed s.c. with saline alone as a control group at the same times. Two hours after the first dose, all rabbits are challenged s.c. with 10⁴ B. anthracis Ames spores and observed for 21 days. At time 0, and each day for 21 days blood serum is obtained. 10 μl of murine serum from each time point is subjected to analysis by the inventive method and EF activity is measured by LC-MS/MS as described in Examples 2-6.

Methods involving conventional biological techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; Current Protocols in Molecular Biology, ed. Ausubel et al., Greene Publishing and Wiley-Interscience, New York, 1992 (with periodic updates); and Short Protocols in Molecular Biology, ed. Ausubel et al., 52 ed., Wiley-Interscience, New York, 2002. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification,” Methods in Enzymology vol. 182, and other volumes in this series; Current Protocols in Protein Science, John Wiley and Sons, New York, N.Y.; and manufacturer's literature on use of protein purification products known to those of skill in the art.

Methods of producing and screening antibodies are illustratively found in Monoclonal Antibodies: Methods and Protocols, Albitar, M, ed., Humana Press, 2010 (ISBN 1617376469); and Antibodies: A Laboratory Manual, Harlos, E, and Lane, D. eds., Cold Spring Harbor Laboratory Press, 1988 (ISBN-10: 0879693142).

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A process for detecting an adenylate cyclase in a sample comprising:
obtaining a sample;
isolating an adenylate cyclase from said sample;
reacting said adenylate cyclase with an adenylate substrate cyclized by said adenylate cyclase to yield a reaction product; and
detecting the presence or absence of said reaction product to detect the presence or absence of said adenylate cyclase in said biological sample.

2. The process of claim 1 wherein said adenylate cyclase is a protein with adenylate cyclase activity, said protein derived from Bacillus anthracis, or Bordetella pertussis.

3. (canceled)

4. The process of claim 1 further comprising quantifying said reaction product.

5. The process of claim 1 wherein said adenylate cyclase is Bacillus anthracis edema factor, Bacillus anthracis edema toxin, Bordetella pertussis adenylate cyclase, protective antigen, or combinations thereof.

6. The process of claim 1, wherein said adenylate cyclase is isolated and concentrated by binding to beads coupled with an antibody specific to the adenylate cyclase or to a molecule bound to the adenylate cyclase.

7. The process of claim 6 wherein said antibody remains bound to said adenylate cyclase during said step of reacting.

8. (canceled)

9. The process of claim 1, wherein said substrate comprises adenosine triphosphate, guanine triphosphate, cytosine triphosphate, or other molecule mimicking a nucleotide triphosphate capable of cyclization or transformation by an adenylate cyclase.

10. The process of claim 1, wherein said substrate is ATP.

11. The process of claim 1, wherein said sample is a biological sample derived from a mammal.

12. The process of claim 1, wherein said sample is derived from a human.

13. The process of claim 1, wherein said sample is whole blood, plasma, serum, pleural fluid, ascites, extracellular fluid, cytosolic fluid, or tissue.

14. The process of claim 1, wherein said adenylate cyclase is anthrax edema factor or anthrax edema toxin and said isolating said anthrax edema factor or edema toxin is performed by adsorption to a solid substrate.

15-16. (canceled)

17. The process of claim 6 wherein said antibody recognizes a Bacillus anthracis protein that is protective antigen, edema factor, edema toxin, or combinations thereof.

18. The process of claim 1, wherein said detecting is by LC-ESI-MS, coupled enzyme assay, continuous enzyme assay, discontinuous enzyme assay, flow cytometry, high-performance liquid chromatography, or combinations thereof.

19. A kit for detecting an antigen from Bacillus anthracis comprising:
a reaction chamber;
magnetic beads coated with an anti-edema factor antibody;
a nucleotide triphosphate substrate cyclizable by said antigen; and
suitable buffers.

20. The kit of claim 19 further comprising:
a second reaction chamber for cyclization of nucleotide triphosphate.

21. The kit of claim 19 further comprising:
magnetic beads coated with an anti-protective antigen antibody.

22. A process for identifying or quantifying Bacillus anthracis edema factor in serum, plasma, or other body fluid comprising:
isolating edema factor from said serum, plasma, or other body fluid on magnetic protein-G or tosyl-activated and anti-edema factor antibody coated beads;
reacting said edema factor with ATP to yield a product;
detecting an amount of said product using mass spectrometry; and
quantifying said edema factor present in said serum, plasma, or other body fluid by comparison of said amount to a standard curve.

23. The process of claim 22 wherein said serum, plasma, or other body fluid is derived from a mammal.

24. The process of claim 22 wherein said serum, plasma, or other body fluid is derived from a human.

25-28. (canceled)
